(19) United States
(12) Patent Application Publication
(10) Pub. No.: US 2023/0086936 A1
(43) Pub. Date: Mar. 23, 2023

(54) COMPOUNDS AND METHODS FOR REDUCING APOE EXPRESSION

(71) Applicant: SynerK Inc., Concord, MA (US)

(72) Inventors: Weiwen JIANG, Winchester, MA (US); Jimmy X. TANG, Southborough, MA (US); Daqing WANG, North Reading, MA (US); Dong YU, Westboro, MA (US)

(21) Appl. No.: 17/929,955

(22) Filed: Sep. 6, 2022

Related U.S. Application Data
(63) Continuation of application No. PCT/US21/20414, filed on Mar. 2, 2021.
(60) Provisional application No. 62/985,570, filed on Mar. 5, 2020.

(51) Int. Cl. C12N 15/113

(52) U.S. Cl. CPC C12N 15/113; C12N 2310/315

(57) ABSTRACT

The invention provides compounds, methods, and pharmaceutical compositions for reducing the amount or activity of Apolipoprotein E (ApoE) mRNA, and in certain embodiments reducing the amount of ApoE protein in a cell or animal, wherein reducing the amount or activity of ApoE would be beneficial.

### RELATED APPLICATIONS

This application is a continuation of International Application No. PCT/US21/20414, which designated the United States and was filed on Mar. 2, 2021, published in English, which claims the benefit of U.S. Provisional Application No. 62/985,570, filed on Mar. 5, 2020. The entire teachings of the above applications are incorporated herein by reference.

### SEQUENCE LISTING

The sequence listing submitted via EFS, in compliance with 37 CFR § 1.52(e)(5), is incorporated herein by reference. The sequence listing XML file submitted via EFS contains the file “4269.3001 US1 SEQ.xml”, created on Sep. 6, 2022, which is 164,835 bytes in size.

### FIELD

Provided are compounds, methods, and pharmaceutical compositions for reducing the amount or activity of apolipoprotein E (ApoE) mRNA in a cell or animal, and in certain instances reducing the amount of ApoE protein in a cell or animal. Such compounds, methods, and pharmaceutical compositions are useful to prevent or ameliorate at least one symptom or hallmark of a liver or a neurological disease. Such liver and neurological diseases include hepatitis B viral infection and Alzheimer's disease.

### BACKGROUND

Apolipoprotein E (ApoE, AD2, APO-E, LDLCQ5, LPG and ApoE4) belongs to a family of lipoproteins that binds to fat. It interacts with low-density lipoprotein receptor (LDLR), essential for the catabolism of triglyceride rich lipoproteins¹. ApoE is primarily synthesized in the liver but has also been found in other parts of the body such as intestine kidneys, brain and spleen. In the brain, ApoE is produced by astrocytes and functions as the primary cholesterol carrier.

ApoE has been implicated in cardiovascular and Alzheimer's diseases^{3,4}. ApoE is also involved in immune regulation⁵. In humans, there are three major isoforms of ApoE, e2, 3 and 4.

ApoE has also been implicated in many viral infections such as herpes simplex virus⁶, human immunodeficiency virus⁷ and hepatitis C virus (HCV)^{8,9}. Recent evidence suggests that APOE is also involved in hepatitis B virus (HBV) infection and production¹⁰. Similar to HCV, ApoE is associated with HBV and is required for efficient viral infection^{8,10}. HBV infection is greatly reduced in ApoE knock liver cells and viral production is also reduced¹⁰. This effect can be reversed by reintroducing ApoE into the cell culture. In addition, ApoE has been associated with progression of HBV-related liver diseases¹¹.

Current available HBV treatment are not curative and HBV vaccine does not offer therapeutic effect for chronically HBV infected individuals¹². Taken together, targeting ApoE may offer a curative approach in treating HBV infection.

### SUMMARY OF THE INVENTION

Provided herein are compounds, methods, and pharmaceutical compositions for reducing the amount or activity of Apolipoprotein E (ApoE) mRNA, and in certain embodiments reducing the amount of ApoE protein in a cell or animal, wherein reducing the amount or activity of ApoE would be beneficial. Certain embodiments are drawn to a method of reducing expression of ApoE in a cell comprising contacting the cell with an oligomeric compounds or modified oligonucleotides as described herein. Certain embodiments are drawn to a method of reducing expression of ApoE in a patient comprising administering an oligomeric compounds or modified oligonucleotides as described herein.

In certain embodiments, the animal can be a transgenic animal or an adeno-associated virus-mediated viral infection animal.

In certain embodiments, compounds useful for reducing expression of ApoE mRNA are oligomeric compounds or modified oligonucleotides. In certain embodiments, the oligomeric compound comprises a modified oligonucleotide.

Also provided are methods useful for ameliorating at least one symptom or hallmark of a liver disease. In certain embodiments, the liver disease is hepatitis B viral infection.

Also provided are methods useful for ameliorating at least one symptom or hallmark of a neurological disease. In certain embodiments, the neurological disease is Alzheimer's.

Such symptoms and hallmarks include viral load, jaundice, fever, liver cirrhosis, liver cancer, cognitive decline, behavioral changes and mood swings.

### BRIEF DESCRIPTION OF THE DRAWING

FIG. 1 shows the effects of modified APOE antisense in human Hep3B cells.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” means a nucleoside comprising a 2’-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5-position. A 5-methyl cytosine is a modified nucleobase.

As used herein, “administering” means providing a pharmaceutical agent to an animal.

As used herein, “animal” means a human or non-human animal.

As used herein, “individual in need thereof” refers to a human or non-human animal selected for treatment or therapy that is in need of such treatment or therapy.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom or hallmark is ataxia, neuropathy, and aggregate formation. In certain embodiments, amelioration of these symptoms results in improved motor function, reduced neuropathy, or reduction in number of aggregates.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases refer to nucleobases that are capable of forming hydrogen bonds with one another.

Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moieties of the nucleosides of the gap of a gapmer are unmodified 2′-deoxyfuranosyl. Thus, the term “MOE gapmer” indicates a gapmer having a sugar motif of 2′-MOE nucleosides in both wings and a gap of 2′-deoxynucleosides. Unless otherwise indicated, a MOE gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid amenable to oligomeric compounds for reducing the amount or activity of the target nucleic acid as demonstrated in the examples hereinbelow.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” is a modified internucleoside linkage in which one of the non-bridging oxygen atoms of a phosphodiester internucleoside linkage is replaced with a sulfur atom.

As used herein, the phrase “inhibiting the expression or activity” refers to a reduction or blockade of the expression or activity relative to the expression of activity in an untreated or control sample and does not necessarily indicate a total elimination of expression or activity.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “MOE” means methoxyethyl. “2′-MOE” means a 2′-OCH₂CH₂OCH₃ group in place of the 2′ OH group of a ribosyl sugar moiety.

As used herein, “motif” means the pattern of unmodified and/or modified sugar moieties, nucleobases, and/or internucleoside linkages, in an oligonucleotide.

As used herein, “mRNA” means an RNA transcript that encodes a protein and includes pre-mRNA and mature mRNA unless otherwise specified.

As used herein, “nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein “pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a different form within an animal or cells thereof. Typically, conversion of a prodrug within the animal is facilitated by the action of an enzymes (e.g., endogenous or viral enzyme) or chemicals present in cells or tissues and/or by physiologic conditions.

As used herein, “OMe” means methoxy. “2′-OMe” means a 2′-OCH₃ group in place of the 2′ OH group of a ribosyl sugar moiety.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “self-complementary” in reference to an oligonucleotide means an oligonucleotide that at least partially hybridizes to itself.

As used herein, “standard cell assay” means the assay described in Example 1 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid” and “target RNA” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to an animal. For example, a therapeutically effective amount improves a symptom of a disease.

As used herein, “treat”, “treatment”, or “treating” refers to administering a compound described herein to effect an alteration or improvement of a disease, disorder, or condition.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

Certain embodiments provide compounds, compositions and methods for reducing Apolipoprotein E (ApoE) mRNA and protein expression. In certain embodiments, the compound is a ApoE specific inhibitor for treating, preventing, or ameliorating a ApoE associated disease. In certain embodiments, the compound is an antisense oligonucleotide targeting ApoE.

| In certain embodiments provided are antisense compounds targeted to a human ApoE | |
|---|---|
| nucleic acid. In certain embodiments, the human ApoE nucleic acid is the sequence | |
| set forth in GENBANK Accession No. NM_001302690.1 | |
| (SEQ ID NO: 1) | |
| 1 | ggatggggag ataagagaag accaggaggg agttaaatag ggaatgggtt gggggcggct |
| 61 | tggtaaatgt gctgggatta ggctgttgca gataatgcaa caaggcttgg aaggctaacc |
| 121 | tgggactggc caatcacagg caggaagatg aaggttctgt gggctgcgtt gctggtcaca |
| 181 | ttcctggcag gatgccaggc caaggtggag caagcggtgg agacagagcc ggagcccgag |
| 241 | ctgcgccagc agaccgagtg gcagagcggc cagcgctggg aactggcact gggtcgcttt |
| 301 | tgggattacc tgcgctgggt gcagacactg tctgagcagg tgcaggagga gctgctcagc |
| 361 | tcccaggtca cccaggaact gagggcgctg atggacgaga ccatgaagga gttgaaggcc |
| 421 | tacaaatcgg aactggagga acaactgacc ccggtggcgg aggagacgcg ggcacggctg |
| 481 | tccaaggagc tgcaggcggc gcaggcccgg ctgggcgcgg acatggagga cgtgtgcggc |
| 541 | cgcctggtgc agtaccgcgg cgaggtgcag gccatgctcg gccagagcac cgaggagctg |
| 601 | cgggtgcgcc tcgcctccca cctgcgcaag ctgcgtaagc ggctcctccg cgatgccgat |
| 661 | gacctgcaga agcgcctggc agtgtaccag gccggggccc gcgagggcgc cgagcgcggc |
| 721 | ctcagcgcca tccgcgagcg cctggggccc ctggtggaac agggccgcgt gcgggccgcc |
| 781 | actgtgggct ccctggccgg ccagccgcta caggagcggg cccaggcctg gggcgagcgg |
| 841 | ctgcgcgcgc ggatggagga gatgggcagc cggacccgcg accgcctgga cgaggtgaag |
| 901 | gagcaggtgg cggaggtgcg cgccaagctg gaggagcagg cccagcagat acgcctgcag |
| 961 | gccgaggcct tccaggcccg cctcaagagc tggttcgagc ccctggtgga agacatgcag |
| 1021 | cgccagtggg ccgggctggt ggagaaggtg caggctgccg tgggcaccag cgccgcccct |
| 1081 | gtgcccagcg acaatcactg aacgccgaag cctgcagcca tgcgacccca cgccaccccg |
| 1141 | tgcctcctgc ctccgcgcag cctgcagcgg gagaccctgt ccccgcccca gccgtcctcc |
| 1201 | tggggtggac cctagtttaa taaagattca ccaagtttca cgcatcaaaa aaaaaaaaaa |
| 1261 | aaaaa |
| In certain embodiments provided are antisense compounds targeted to a mouse ApoE | |
| nucleic acid. In certain embodiments, the mouse ApoE nucleic acid is the sequence | |
| set forth in GENBANK Accession No. NM_001305843.1 | |
| (SEQ ID NO: 2) | |
| 1 | tttcctctgc cctgctgtga agggggagag aacaacccgc ctcgtgacag ggggctggca |
| 61 | cagcccgccc tagccctgag gagggggcgg gacaggggga gtcctataat tggaccggtc |
| 121 | tgggatccga tcccctgctc agaccctgga ggctaaggac ttgtttcgga aggagctgga |
| 181 | gagggagctg gaatttttgg cagcggatcc accccggggt gccgagatag cgaactcggc |
| 241 | aaggggagac tggccaatca caattgcgaa gatgaaggct ctgtgggccg tgctgttggt |
| 301 | cacattgctg acaggatgcc tagccgaggg agagccggag gtgacagatc agctcgagtg |
| 361 | gcaaagcaac caaccctggg agcaggccct gaaccgcttc tgggattacc tgcgctgggt |
| 421 | gcagacgctg tctgaccagg tccaggaaga gctgcagagc tcccaagtca cacaagaact |
| 481 | gacggcactg atggaggaca ctatgacgga agtaaaggct tacaaaaagg agctggagga |
| 541 | acagctgggt ccagtggcgg aggagacacg ggccaggctg ggcaaagagg tgcaggcggc |
| 601 | acaggcccga ctcggagccg acatggagga tctacgcaac cgactcgggc agtaccgcaa |
| 661 | cgaggtgcac accatgctgg gccagagcac agaggagata cgggcgcggc tctccacaca |
| 721 | cctgcgcaag atgcgcaagc gcttgatgcg ggatgccgag gatctgcaga agcgcctagc |
| 781 | tgtgtacaag gcaggggcac gcgagggcgc cgagcgcggt gtgagtgcca tccgtgagcg |
| 841 | cctggggcct ctggtggagc aaggtcgcca gcgcactgcc aacctaggcg ctggggccgc |
| 901 | ccagcctctg cgcgatcgcg cccaggcttt tggtgaccgc atccgagggc ggctggagga |
| 961 | agtgggcaac caggcccgtg accgcctaga ggaggtgcgt gagcacatgg aggaggtgcg |
| 1021 | ctccaagatg gaggaacaga cccagcaaat acgcctgcag gcggagatct tccaggcccg |
| 1081 | cctcaagggc tggttcgagc caatagtgga agacatgcat cgccagtggg caaacctgat |
| 1141 | ggagaagata caggcctctg tggctaccaa ccccatcatc accccagtgg cccaggagaa |
| 1201 | tcaatgagta tccttctcct gtcctgcaac aacatccata tccagccagg tggccctgtc |
| 1261 | tcaagcacct ctctggccct ctggtggccc ttgcttaata aagattctcc gagcacattc |
| 1321 | tgagtctctg tgagtgattc caaaaaaaaa aaaaaaaa |

Certain embodiments provide a compound targeting ApoE, wherein the compound comprises of 12 to 30 linked nucleosides. In certain embodiments, the compound consists of 15 to 30, 18 to 24, 19 to 22, 13 to 25, 14 to 25, or 15 to 25 linked nucleosides. In certain embodiments, the compound comprises at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or 30 linked nucleosides. In certain embodiments, the compound consists of 20 linked nucleosides. In certain embodiments, the compound consists of 21 linked nucleosides.

A synthetic oligonucleotide compound comprising 12 to 30 phosphorothioate linked nucleotides having at least 12 contiguous nucleobases complementary to an equal length portion of SEQ ID NO: 1. Certain embodiments provide a compound targeting ApoE, wherein the compound consists of 12 to 30 linked nucleosides and has a nucleobase sequence comprising at least 8, at least 9, at least 10, at least 11, at least 12, at least 13, at least 14, at least 15, at least 16, at least 17, at least 18, at least 19, at least 20, at least 21, or 22 contiguous nucleobases of any of the nucleobase sequences of SEQ ID NO: 1. A synthetic oligonucleotide compound comprising 12 to 30 phosphorothioate linked nucleotides having at least 21 contiguous nucleobases complementary to an equal length portion of SEQ ID NO: 1.

A synthetic oligonucleotide compound comprising 12 to 30 phosphorothioate linked nucleotides wherein the nucleobase sequence of the compound is at least 80% complementary to an equal length portion of SEQ ID NO: 1.

In embodiments, the invention provides an oligomeric compound, comprising a modified oligonucleotide consisting of 12 to 50 linked nucleosides wherein the nucleobase sequence of the modified oligonucleotide is at least 90% complementary to an equal length portion of a apolipoprotein E (ApoE) nucleic acid, and wherein the modified oligonucleotide comprises at least one modification selected from a modified sugar, a sugar surrogate, and a modified internucleoside linkage.

In any embodiment herein, the invention provides an oligomeric compound comprising a modified oligonucleotide consisting of 10-30 linked nucleosides and having a nucleobase sequence comprising at least 12, at least 13, at least 14, at least 15, or at least 16 contiguous nucleobases of any of SEQ ID NO: 87-170.

In any embodiment herein of the oligomeric compound, the modified oligonucleotide has a nucleobase sequence that is at least 80%, 85%, 90%, 95%, or 100% complementary to the nucleobase sequence of SEQ ID NO: 1, when measured across the entire nucleobase sequence of the modified oligonucleotide.

In certain embodiments, the ApoE specific inhibitor is a synthetic oligonucleotide compound comprising 12 to 30 linked nucleotides wherein the nucleobase sequence of the compound is at least 80% complementary to an equal length portion of nucleobases 200-600 or 900-1000 of SEQ ID NO: 1.

In certain embodiments, the gene silencing compound targets anywhere within the region spanning from nucleobase 200 to 400, 240 to 440, 300 to 500, 400-600, or 925 to 975 of SEQ ID NO: 1.

In certain embodiments, the gene silencing compound targets anywhere within the region spanning from nucleobase 200 to 300, 300 to 400, 400 to 500, 500-600, or 930 to 960 of SEQ ID NO: 1.

In certain embodiments, the gene silencing compound targets anywhere within the region spanning from nucleobase 200 to 300 of SEQ ID NO: 1.

In certain embodiments, the gene silencing compound targets anywhere within the region spanning from nucleobase 225 to 275 of SEQ ID NO: 1.

In certain embodiments, the gene silencing compound targets anywhere within the region spanning from nucleobase 300 to 400 of SEQ ID NO: 1.

In certain embodiments, the gene silencing compound targets anywhere within the region spanning from nucleobase 300 to 360 of SEQ ID NO: 1.

In certain embodiments, the gene silencing compound targets anywhere within the region spanning from nucleobase 400 to 500 of SEQ ID NO: 1.

In certain embodiments, the gene silencing compound targets anywhere within the region spanning from nucleobase 400 to 450 of SEQ ID NO: 1.

In certain embodiments, the gene silencing compound targets anywhere within the region spanning from nucleobase 500 to 600 of SEQ ID NO: 1.

In certain embodiments, the gene silencing compound targets anywhere within the region spanning from nucleobase 550 to 600 of SEQ ID NO: 1.

In certain embodiments, the gene silencing compound targets anywhere within the region spanning from nucleobase 900 to 1000 of SEQ ID NO: 1.

In certain embodiments, the gene silencing compound targets anywhere within the region spanning from nucleobase 925 to 975 of SEQ ID NO: 1.

In certain embodiments, the gene silencing compound targets anywhere within the region spanning from nucleobase 930 to 960 of SEQ ID NO: 1.

In any embodiment herein, the modified oligonucleotide comprises at least one modified nucleoside.

In any embodiment herein, the modified oligonucleotide comprises at least one modified nucleoside comprising a modified sugar moiety.

In any embodiment herein, the modified oligonucleotide comprises at least one modified nucleoside comprising a bicyclic sugar moiety.

In any embodiment herein, the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.

In any embodiment herein, the modified oligonucleotide comprises at least one modified nucleoside comprising a modified non-bicyclic sugar moiety.

In any embodiment herein, the modified oligonucleotide comprises at least one modified nucleoside comprising a non-bicyclic sugar moiety comprising a 2′-MOE or 2′-OMe. In any embodiment herein, the modified oligonucleotide comprises at least one modified nucleoside comprising a sugar surrogate.

In any embodiment herein, the modified oligonucleotide comprises at least one modified nucleoside comprising a sugar surrogate selected from morpholino and PNA.

In any embodiment herein, the modified oligonucleotide has a sugar motif comprising:

a 5′-region consisting of 1-6 linked 5′-nucleosides;

a central region consisting of 6-15 linked central region nucleosides; and

a 3′-region consisting of 1-6 linked 3′-region nucleosides;

wherein each of the 5′-region nucleosides and each of the 3′-region nucleosides comprises a modified sugar moiety and each of the central region nucleosides comprises an unmodified DNA sugar moiety.

In any embodiment herein, the modified oligonucleotide comprises at least one modified internucleoside linkage.

In any embodiment herein, wherein each internucleoside linkage of the modified oligonucleotide is a modified internucleoside linkage.

In any embodiment herein, wherein at least one internucleoside linkage is a phosphorothioate internucleoside linkage.

In any embodiment herein, wherein the modified oligonucleotide comprises at least one phosphodiester internucleoside linkage.

In any embodiment herein, wherein each internucleoside linkage is either a phosphodiester internucleoside linkage or a phosphorothioate internucleoside linkage.

In any embodiment herein, the modified oligonucleotide comprises at least one modified nucleobase.

In any embodiment herein, wherein the modified nucleobase is a 5-methylcytosine.

In any embodiment herein, wherein the modified oligonucleotide consists of 12-22, 12-22, 14-22, 16-22, or 18-22 linked nucleosides.

In any embodiment herein, wherein the modified oligonucleotide consists of 16, 17, 18, 19, 20, 21, or 22 linked nucleosides.

In any embodiment herein, wherein the modified oligonucleotide consists of 21 linked nucleosides.

In any embodiment herein, consisting of the modified oligonucleotide.

In any embodiment herein, comprising a conjugate group comprising a conjugate moiety and a conjugate linker.

In any embodiment herein, wherein the conjugate group comprises a GalNAc cluster comprising 1-3 GalNAc ligands.

In any embodiment herein, wherein the conjugate linker consists of a single bond.

In any embodiment herein, wherein the conjugate linker is cleavable.

In any embodiment herein, wherein the conjugate linker comprises 1-3 linker-nucleosides.

In any embodiment herein, wherein the conjugate group is attached to the modified oligonucleotide at the 5′-end of the modified oligonucleotide.

In any embodiment herein, wherein the conjugate group is attached to the modified oligonucleotide at the 3′-end of the modified oligonucleotide.

In any embodiment herein, comprising a terminal group.

In any embodiment herein, wherein the oligomeric compound is a singled-stranded oligomeric compound.

In any embodiment herein, wherein the oligomeric compound does not comprise linker-nucleosides.

In embodiments, the invention provides an antisense compound comprising or consisting of an oligomeric compound of any of embodiments herein.

In any embodiment herein, the invention provides a modified oligonucleotide consisting of 10-30 linked nucleosides and having a nucleobase sequence comprising at least 12, at least 13, at least 14, at least 15, or at least 16 contiguous nucleobases of any of SEQ ID NO: 87-170.

In any embodiment herein, the invention provides an oligomeric compound comprising a modified oligonucleotide consisting of 10-30 linked nucleosides and having a nucleobase sequence comprising a portion of at least 8, at least 9, at least 10, at least 11, at least 12, at least 13, or at least 14 contiguous nucleobases 100% complementary to an equal length portion of hotspot of SEQ ID NO: 1. In embodiments, the nucleobase sequence of the modified oligonucleotide is at least 90% complementary to SEQ ID NO: 1 as measured over the entirety of the modified oligonucleotide.

In embodiments, the invention provides a pharmaceutical composition comprising an oligomeric compound of any of embodiments herein, or a modified oligonucleotide described herein and a pharmaceutically acceptable carrier or diluent.

In embodiments, the invention provides a method comprising administering to an animal a pharmaceutical composition described herein.

Certain embodiments are drawn to a method of reducing expression of ApoE in a cell comprising contacting the cell with an oligomeric compounds or modified oligonucleotides as described herein.

Certain embodiments are drawn to a method of reducing expression of ApoE in a patient comprising administering an oligomeric compounds or modified oligonucleotides as described herein.

In embodiments, the invention provides a method of inhibiting expression of ApoE in a cell, comprising contacting a cell with an oligomeric compounds or modified oligonucleotides as described herein, and thereby inhibiting expression of ApoE.

In embodiments, the invention provides a method of inhibiting expression of ApoE in a patient, comprising administering an oligomeric compounds or modified oligonucleotides as described herein, and thereby inhibiting expression of ApoE.

In embodiments, the invention provides a method of treating a disease associated with ApoE comprising administering to an individual having or at risk for developing a disease associated with ApoE a therapeutically effective amount of a pharmaceutical composition described herein; and thereby treating the disease associated with ApoE.

In any embodiment herein, the disease associated with ApoE is a liver disease. In any embodiment herein, the liver disease is hepatitis B viral infection.

In any embodiment herein, the disease associated with ApoE is a neurological disease. In any embodiment herein, the neurological disease is Alzheimer's.

In embodiments, the invention provides a chirally enriched population of oligomeric compounds of any of embodiments herein wherein the population is enriched for modified oligonucleotides comprising at least one particular phosphorothioate internucleoside linkage having a particular stereochemical configuration.

In any embodiment herein, wherein the population is enriched for modified oligonucleotides comprising at least one particular phosphorothioate internucleoside linkage having the (Sp) configuration.

In any embodiment herein, wherein the population is enriched for modified oligonucleotides comprising at least one particular phosphorothioate internucleoside linkage having the (Rp) configuration.

In any embodiment herein, wherein the population is enriched for modified oligonucleotides having a particular, independently selected stereochemical configuration at each phosphorothioate internucleoside linkage.

In any embodiment herein, wherein the population is enriched for modified oligonucleotides having the (Sp) configuration at each phosphorothioate internucleoside linkage.

In any embodiment herein, wherein the population is enriched for modified oligonucleotides having the (Rp) configuration at each phosphorothioate internucleoside linkage.

In any embodiment herein, wherein the population is enriched for modified oligonucleotides having the (Rp) configuration at one particular phosphorothioate internucleoside linkage and the (Sp) configuration at each of the remaining phosphorothioate internucleoside linkages.

In any embodiment herein, wherein the population is enriched for modified oligonucleotides having at least 3 contiguous phosphorothioate internucleoside linkages in the Sp, Sp, and Rp configurations, in the 5′ to 3′ direction.

In any embodiment herein, wherein all of the phosphorothioate internucleoside linkages of the modified oligonucleotide are stereorandom.

In any embodiment herein, wherein the population is enriched for modified oligonucleotides comprising at least one particular phosphorothioate internucleoside linkage having a particular stereochemical configuration.

In any embodiment herein, wherein the population is enriched for modified oligonucleotides comprising at least one particular phosphorothioate internucleoside linkage having the (Sp) configuration.

In any embodiment herein, wherein the population is enriched for modified oligonucleotides comprising at least one particular phosphorothioate internucleoside linkage having the (Rp) configuration.

In any embodiment herein, wherein the population is enriched for modified oligonucleotides having a particular, independently selected stereochemical configuration at each phosphorothioate internucleoside linkage.

In any embodiment herein, wherein the population is enriched for modified oligonucleotides having the (Sp) configuration at each phosphorothioate internucleoside linkage.

In any embodiment herein, wherein the population is enriched for modified oligonucleotides having the (Rp) configuration at each phosphorothioate internucleoside linkage.

In any embodiment herein, wherein the population is enriched for modified oligonucleotides having the (Rp) configuration at one particular phosphorothioate internucleoside linkage and the (Sp) configuration at each of the remaining phosphorothioate internucleoside linkages.

In any embodiment herein, wherein the population is enriched for modified oligonucleotides having at least 3 contiguous phosphorothioate internucleoside linkages in the Sp, Sp, and Rp configurations, in the 5′ to 3′ direction.

In any embodiment herein, wherein all of the phosphorothioate internucleoside linkages of the modified oligonucleotide are stereorandom.

In certain embodiments, provided herein are oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al., Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

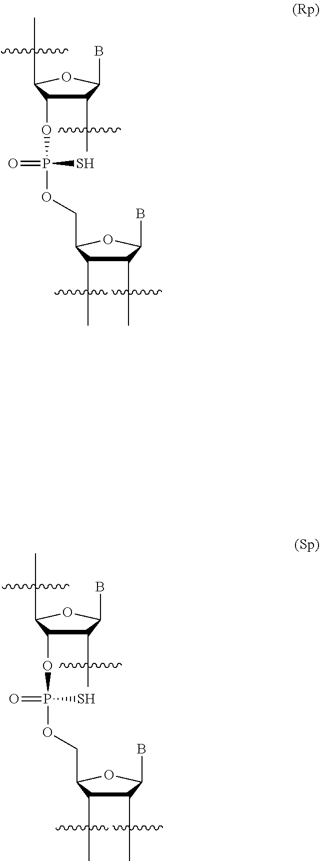

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4(3′-CH₂—N(H)—C(═O)-5′), formacetal (3′-O—CH₂—O-5′), methoxypropyl, and thioformacetal (3′-5-CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Modified nucleosides comprise a modified sugar moiety or a modified nucleobase or both a modified sugar moiety and a modified nucleobase.

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO2), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al, WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al, U.S. Pat. No. 7,569,686, Swayze et al., U.S. Pat. No. 7,741,457, and Swayze et al, U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)—O-2′ and analogs thereof (see, e.g., Prakash et al, U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4, —C(RₐR_{b})—O—N(R)-2′, 4′-CH₂—O—N(R)-2′, and 4′-CH₂—N(R)—O-2′, wherein each R, Rₐ and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein:

x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-Cao aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al, Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J Am. Chem. Soc, 20017, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

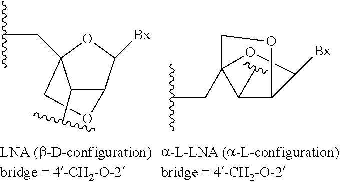

α-L-methyleneoxy (4′-CH₂-0-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

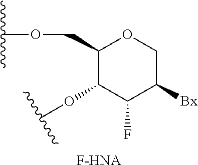

(“F-HNA”, see e.g., Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

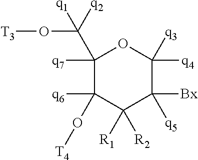

wherein, independently, for each of said modified THP nucleoside:

Bx is a nucleobase moiety;

T3 and T4 are each, independently, an internucleoside linking group linking the modified THP nucleoside to the remainder of an oligonucleotide or one of T3 and T4 is an internucleoside linking group linking the modified THP nucleoside to the remainder of an oligonucleotide and the other of T3 and T4 is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

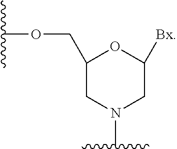

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer independently comprise 1-6 nucleosides. In certain embodiments, the wings of a gapmer independently comprise 1-5 nucleosides. In certain embodiments, the wings of a gapmer comprise the same number of nucleosides. In certain embodiments, the wings of a gapmer comprise 4 nucleosides. In certain embodiments, each nucleoside of each wing of a gapmer is a modified nucleoside.

In certain embodiments, the gap of a gapmer comprises 7-23 nucleosides. In certain embodiments, the gap of a gapmer comprises 7-17 nucleosides. In certain embodiments, the gap of a gapmer comprises 9-14 nucleosides. In certain embodiments, the gap of a gapmer comprises 7-23 nucleosides. In certain embodiments, the gap of a gapmer comprises 9 nucleosides. In certain embodiments, the gap of a gapmer comprises 10 nucleosides. In certain embodiments, the gap of a gapmer comprises 11 nucleosides. In certain embodiments, the gap of a gapmer comprises 13 nucleosides. In certain embodiments, the gap of a gapmer comprises 14 nucleosides. In certain embodiments, the gap of a gapmer comprises 17 nucleosides. In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

Herein, the lengths (number of nucleosides) of the three regions of a gapmer may be provided using the notation [# of nucleosides in the 5′-wing]—[# of nucleosides in the gap]—[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in the wings and the gap nucleosides comprise unmodified deoxynucleosides sugars. Thus, a 5-11-5 MOE or OMe gapmer consists of 5 linked MOE or OMe modified nucleosides in the 5′-wing, 11 linked deoxynucleosides in the gap, and 5 linked MOE or OMe nucleosides in the 3′-wing.

In certain embodiments, modified oligonucleotides are 4-13-4 MOE or OMe gapmers. In certain embodiments, modified oligonucleotides are 5-11-5 MOE or OME gapmers. In certain embodiments, modified oligonucleotides are 3-15-3 BNA gapmers. In certain embodiments, modified oligonucleotides are 3-15-3 LNA gapmers.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification. In certain embodiments, the uniformly modified sugar motif is 12 to 30 nucleosides in length. In certain embodiments, each nucleoside of the uniformly modified sugar motif is a 2′-substituted nucleoside, a sugar surrogate, or a bicyclic nucleoside. In certain embodiments, each nucleoside of the uniformly modified sugar motif comprises either a 2′-OCH₂CH₂OCH₃ group or a 2′-OCH₃ group. In certain embodiments, modified oligonucleotides having at least one fully modified sugar motif may also have at least 1, at least 2, at least 3, or at least 4 2′-deoxynucleosides.

In certain embodiments, each nucleoside of the entire modified oligonucleotide comprises a modified sugar moiety (fully modified oligonucleotide). In certain embodiments, a fully modified oligonucleotide comprises different 2′-modifications. In certain embodiments, each nucleoside of a fully modified oligonucleotide is a 2′-substituted nucleoside, a sugar surrogate, or a bicyclic nucleoside. In certain embodiments, each nucleoside of a fully modified oligonucleotide comprises either a 2′-OCH₂CH₂OCH₃ group and at least one 2′-OCH₃ group.

In certain embodiments, each nucleoside of a fully modified oligonucleotide comprises the same 2′-modification (uniformly modified oligonucleotide). In certain embodiments, each nucleoside of a uniformly modified oligonucleotide is a 2′-substituted nucleoside, a sugar surrogate, or a bicyclic nucleoside. In certain embodiments, each nucleoside of a uniformly modified oligonucleotide comprises either a 2′-OCH₂CH₂OCH₃ group or a 2′-OCH₃ group.

In certain embodiments, modified oligonucleotides comprise at least 12, at least 13, at least 14, at least 15, at least 16, at least 17, at least 18, at least 19, at least 20, at least 21, at least 22, at least 23, at least 24, at least 25, or at least 26 nucleosides comprising a modified sugar moiety. In certain embodiments, each nucleoside of a modified oligonucleotide is a 2′-substituted nucleoside, a sugar surrogate, a bicyclic nucleoside, or a 2′-deoxynucleoside. In certain embodiments, each nucleoside of a modified oligonucleotide comprises a 2′-OCH₂CH₂OCH₃ group, a 2′-H(H) deoxyribosyl sugar moiety, or a cEt modified sugar.

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular sterochemical configuration.

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

In certain embodiments, the invention provides oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target mRNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 1 1, 12, 13, 14, 15, 16, 17, 18, 19, 20, 21, 22, 23, 24, 25, 26, 27, 28, 29, and 30; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycerol-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

In certain embodiments, oligomeric compounds comprise one or more terminal groups. In certain such embodiments, oligomeric compounds comprise a stabilized 5′-phophate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

In certain embodiments, oligomeric compounds are capable of hybridizing to a target nucleic acid, resulting in at least one antisense activity; such oligomeric compounds are antisense compounds. In certain embodiments, antisense compounds have antisense activity when they reduce or inhibit the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain such embodiments, the target non-coding RNA is selected from: a long non-coding RNA, a short non-coding RNA, an intronic RNA molecule.

It is possible to introduce mismatch bases without eliminating activity (see e.g., Gautschi et al. (J. Natl. Cancer Inst. 93:463-471, March 2001). In certain embodiments, oligomeric compounds comprise oligonucleotides that are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligomeric compound comprising an oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

In certain embodiments, oligomeric compounds comprise or consist of an oligonucleotide comprising a region that is complementary to a target nucleic acid, wherein the target nucleic acid is apolipoprotein E (ApoE). In certain embodiments, human ApoE nucleic acid has the sequence set forth in SEQ ID NO: 1 (GENBANK Accession No: NM_001302690.1). In certain embodiments, mouse ApoE nucleic acid has the sequence set forth in SEQ ID NO: 2 (GENBANK Accession No: NM_001305843.1).

In certain embodiments, contacting a cell with an oligomeric compound complementary to SEQ ID NO: 1 or 2 reduces the amount of ApoE mRNA, and in certain embodiments reduces the amount of ApoE protein. In certain embodiments, contacting a cell in an animal with an oligomeric compound complementary to SEQ ID NO: 1 or 2 ameliorate one or more symptoms or hallmarks of hepatitis B viral infection or Alzheimer's disease.

In certain embodiments, modified oligonucleotides are complementary to a hotspot of SEQ ID NO: 1. In certain embodiments, such modified oligonucleotides are 21 nucleobases in length.

In certain embodiments, such modified oligonucleotides are uniformly MOE or Ome modified oligonucleotides. In certain embodiments, the nucleosides of the modified oligonucleotides are linked by phosphorothioate internucleoside linkages.

In certain embodiments, such modified oligonucleotides are gapmers. In certain such embodiments, the gapmers are 5-11-5 MOE or OMe gapmers. In certain such embodiments, the gapmers are 4-13-4 MOE or OMe gapmers. In certain such embodiments, the gapmers are 5-11-5 MOE gapmers. In certain such embodiments, the gapmers are 5-11-5 OMe gapmers. In certain such embodiments, the gapmers are 4-13-4 MOE gapmers. In certain such embodiments, the gapmers are 4-13-4 OMe gapmers.

In certain embodiments, the nucleosides of the modified oligonucleotides are linked by mixed phosphodiester (“o”) and phosphorothioate (“s”) internucleoside linkages. In certain embodiments, modified oligonucleotides complementary to a hotspot of SEQ ID NO: 1 achieve at least 40% reduction of ApoE mRNA in vitro in the standard cell assay.

In certain embodiments, oligomeric compounds comprise or consist of an oligonucleotide comprising a region that is complementary to a target nucleic acid, wherein the target nucleic acid is expressed in a pharmacologically relevant tissue.

In certain embodiments, described herein are pharmaceutical compositions comprising one or more oligomeric compounds or a salt thereof. In certain embodiments, the pharmaceutical composition comprises a pharmaceutically acceptable diluent or carrier. In certain embodiments, a pharmaceutical composition comprises a sterile saline solution and one or more oligomeric compound. In certain embodiments, a pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations.

Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal, intracerebroventricular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “ATmCGAUCG,” wherein mC indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, all tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effect of Modified Oligonucleotides on Human ApoE In Vitro, Single Dose

Modified oligonucleotides complementary to an ApoE nucleic acid can be designed and tested for their effect on ApoE mRNA in vitro. The modified oligonucleotides can be tested in a series of experiments that had similar culture conditions.

For example, cultured HepG2 cells at a density of 20,000 cells per well can be transfected using electroporation or lipid transfection with a 2,000 nM concentration of modified oligonucleotide. After a treatment period of approximately 24 to 48 hours, RNA is isolated from the cells and ApoE mRNA levels are measured by quantitative real-time PCR. ApoE mRNA levels are adjusted according to total RNA content. Results can be presented as percent reduction of the amount of ApoE mRNA, relative to untreated control cells. Additional assays may be used to measure the potency and efficacy of these oligonucleotides.

The modified oligonucleotides in the table below can be uniformly modified oligonucleotides. The oligonucleotides can 21 nucleobases in length and each nucleoside can have a 2′substitution or modification as described herein.

The modified oligonucleotides in the table below can also be designed as gapmers. The gapmers can be 21 nucleosides in length, wherein the central gap segment comprises 11 or 13 2′-deoxynucleosides and is flanked by wing segments on both the 5′ end and on the 3′ end comprising four or five nucleosides each. Each nucleoside in the 5′ wing segment and each nucleoside in the 3′ wing segment comprises a 2′modification. The modified oligonucleotides in the table below can also designed as 5-11-5 or 4-13-4 gapmers.

In embodiments, some or all cytosine residues throughout each modified oligonucleotide can be 5-methylcytosines.

In embodiments, each internucleoside linkage is a phosphorothioate internucleoside linkage.

In embodiments, the internucleoside linkages are mixed phosphodiester and phosphorothioate linkages.

“Start site” indicates the 5′-most nucleoside to which the modified oligonucleotide is targeted in the human gene sequence. “Stop site” indicates the 3′-most nucleoside to which the modified oligonucleotide is targeted human gene sequence.

Each modified oligonucleotide listed in Table 1 is targeted to the human ApoE mRNA sequence designated herein as SEQ ID NO: 1.

TABLE 1

| | Seq ID | Start | Stop | | Seq ID |
|---|---|---|---|---|---|
| Target sequence | No: | site | site | Antisense (5′-3′) | No: |
| ggatggggagataagagaaga | 3 | 1 | 21 | TCTTCTCTTATCTCCCCATCC | 87 |
| gggagataagagaagaccagg | 4 | 6 | 26 | CCTGGTCTTCTCTTATCTCCC | 88 |
| ataagagaagaccaggaggga | 5 | 11 | 31 | TCCCTCCTGGTCTTCTCTTAT | 89 |
| agaagaccaggagggagttaa | 6 | 16 | 36 | TTAACTCCCTCCTGGTCTTCT | 90 |
| gaagaccaggagggagttaaa | 7 | 17 | 37 | TTTAACTCCCTCCTGGTCTTC | 91 |
| accaggagggagttaaatagg | 8 | 21 | 41 | CCTATTTAACTCCCTCCTGGT | 92 |
| gagggagttaaatagggaatg | 9 | 26 | 46 | CATTCCCTATTTAACTCCCTC | 93 |
| agttaaatagggaatgggttg | 10 | 31 | 51 | CAACCCATTCCCTATTTAACT | 94 |
| aatagggaatgggttgggggc | 11 | 36 | 56 | GCCCCCAACCCATTCCCTATT | 95 |
| ggaatgggttgggggcggctt | 12 | 41 | 61 | AAGCCGCCCCCAACCCATTCC | 96 |
| cggcttggtaaatgtgctggg | 13 | 46 | 66 | CCCAGCACATTTACCAAGCCG | 97 |
| tggtaaatgtgctgggattag | 14 | 61 | 81 | CTAATCCCAGCACATTTACCA | 98 |
| aatgtgctgggattaggctgt | 15 | 66 | 86 | ACAGCCTAATCCCAGCACATT | 99 |
| gctgggattaggctgttgcag | 16 | 71 | 91 | CTGCAACAGCCTAATCCCAGC | 100 |
| ggattaggctgttgcagataa | 17 | 75 | 95 | TTATCTGCAACAGCCTAATCC | 101 |
| gattaggctgttgcagataat | 18 | 76 | 96 | ATTATCTGCAACAGCCTAATC | 102 |
| ggctgttgcagataatgcaac | 19 | 81 | 101 | GTTGCATTATCTGCAACAGCC | 103 |
| ttgcagataatgcaacaaggc | 20 | 86 | 106 | GCCTTGTTGCATTATCTGCAA | 104 |
| gataatgcaacaaggcttgga | 21 | 91 | 111 | TCCAAGCCTTGTTGCATTATC | 105 |
| tgcaacaaggcttggaaggct | 22 | 96 | 116 | AGCCTTCCAAGCCTTGTTGCA | 106 |
| caaggcttggaaggctaacct | 23 | 101 | 121 | AGGTTAGCCTTCCAAGCCTTG | 107 |
| cttggaaggctaacctgggac | 24 | 106 | 126 | GTCCCAGGTTAGCCTTCCAAG | 108 |
| aaggctaacctgggactggcc | 25 | 111 | 131 | GGCCAGTCCCAGGTTAGCCTT | 109 |
| taacctgggactggccaatca | 26 | 116 | 136 | TGATTGGCCAGTCCCAGGTTA | 110 |
| tgggactggccaatcacaggc | 27 | 121 | 141 | GCCTGTGATTGGCCAGTCCCA | 111 |
| ctggccaatcacaggcaggaa | 28 | 126 | 146 | TTCCTGCCTGTGATTGGCCAG | 112 |
| caatcacaggcaggaagatga | 29 | 131 | 151 | TCATCTTCCTGCCTGTGATTG | 113 |
| acaggcaggaagatgaaggtt | 30 | 136 | 156 | AACCTTCATCTTCCTGCCTGT | 114 |
| gcaggaagatgaaggttctgt | 31 | 140 | 160 | ACAGAACCTTCATCTTCCTGC | 115 |
| caggaagatgaaggttctgtg | 32 | 141 | 161 | CACAGAACCTTCATCTTCCTG | 116 |
| gaagatgaaggttctgtgggc | 33 | 144 | 164 | GCCCACAGAACCTTCATCTTC | 117 |
| agatgaaggttctgtgggctg | 34 | 146 | 166 | CAGCCCACAGAACCTTCATCT | 118 |
| gaaggttctgtgggctgcgtt | 35 | 150 | 179 | AACGCAGCCCACAGAACCTTC | 119 |
| gcgttgctggtcacattcctg | 36 | 166 | 186 | CAGGAATGTGACCAGCAACGC | 120 |
| gctggtcacattcctggcagg | 37 | 171 | 191 | CCTGCCAGGAATGTGACCAGC | 121 |
| tcacattcctggcaggatgcc | 38 | 176 | 196 | GGCATCCTGCCAGGAATGTGA | 122 |
| ttcctggcaggatgccaggcc | 39 | 181 | 201 | GGCCTGGCATCCTGCCAGGAA | 123 |
| ggcaggatgccaggccaaggt | 40 | 186 | 206 | ACCTTGGCCTGGCATCCTGCC | 124 |
| gatgccaggccaaggtggagc | 41 | 191 | 211 | GCTCCACCTTGGCCTGGCATC | 125 |
| caggccaaggtggagcaagcg | 42 | 196 | 216 | CGCTTGCTCCACCTTGGCCTG | 126 |
| ggtggagacagagccggagcc | 43 | 216 | 236 | GGCTCCGGCTCTGTCTCCACC | 127 |
| ctgcgccagcagaccgagtgg | 44 | 241 | 261 | CCACTCGGTCTGCTGGCGCAG | 128 |
| ccagcagaccgagtggcagag | 45 | 246 | 266 | CTCTGCCACTCGGTCTGCTGG | 129 |
| gccagcgctgggaactggcac | 46 | 269 | 289 | GTGCCAGTTCCCAGCGCTGGC | 130 |
| ctgggaactggcactgggtcg | 47 | 276 | 296 | CGACCCAGTGCCAGTTCCCAG | 131 |
| gcttttgggattacctgcgct | 48 | 296 | 316 | AGCGCAGGTAATCCCAAAAGC | 132 |
| tgggattacctgcgctgggtg | 49 | 301 | 321 | CACCCAGCGCAGGTAATCCCA | 133 |
| ctgggtgcagacactgtctga | 50 | 315 | 335 | TCAGACAGTGTCTGCACCCAG | 134 |
| tgggtgcagacactgtctgag | 51 | 316 | 336 | CTCAGACAGTGTCTGCACCCA | 135 |
| gcagacactgtctgagcaggt | 52 | 321 | 341 | ACCTGCTCAGACAGTGTCTGC | 136 |
| cactgtctgagcaggtgcagg | 53 | 326 | 346 | CCTGCACCTGCTCAGACAGTG | 137 |
| tctgagcaggtgcaggaggag | 54 | 331 | 351 | CTCCTCCTGCACCTGCTCAGA | 138 |
| gcaggtgcaggaggagctgct | 55 | 336 | 356 | AGCAGCTCCTCCTGCACCTGC | 139 |
| tgcaggaggagctgctcagct | 56 | 341 | 361 | AGCTGAGCAGCTCCTCCTGCA | 140 |
| gaggagctgctcagctcccag | 57 | 346 | 366 | CTGGGAGCTGAGCAGCTCCTC | 141 |
| gctgctcagctcccaggtcac | 58 | 351 | 371 | GTGACCTGGGAGCTGAGCAGC | 142 |
| tcagctcccaggtcacccagg | 59 | 356 | 376 | CCTGGGTGACCTGGGAGCTGA | 143 |
| tcccaggtcacccaggaactg | 60 | 361 | 381 | CAGTTCCTGGGTGACCTGGGA | 144 |
| aggtcacccaggaactgaggg | 61 | 365 | 385 | CCCTCAGTTCCTGGGTGACCT | 145 |
| ggacgagaccatgaaggagtt | 62 | 393 | 413 | AACTCCTTCATGGTCTCGTCC | 146 |
| ccatgaaggagttgaaggcct | 63 | 401 | 421 | AGGCCTTCAACTCCTTCATGG | 147 |
| gaaggagttgaaggcctacaa | 64 | 405 | 425 | TTGTAGGCCTTCAACTCCTTC | 148 |
| aaggagttgaaggcctacaaa | 65 | 406 | 426 | TTTGTAGGCCTTCAACTCCTT | 149 |
| gttgaaggcctacaaatcgga | 66 | 411 | 431 | TCCGATTTGTAGGCCTTCAAC | 150 |
| aactggaggaacaactgaccc | 67 | 431 | 451 | GGGTCAGTTGTTCCTCCAGTT | 151 |
| ggaggaacaactgaccccggt | 68 | 435 | 455 | ACCGGGGTCAGTTGTTCCTCC | 152 |
| ctgaccccggtggcggaggac | 69 | 445 | 465 | GTCCTCCGCCACCGGGGTCAG | 153 |
| ggctgtccaaggagctgcagg | 70 | 476 | 496 | CCTGCAGCTCCTTGGACAGCC | 154 |
| aggtgcaggccatgctcggcc | 71 | 563 | 583 | GGCCGAGCATGGCCTGCACCT | 155 |
| gccatgctcggccagagcacc | 72 | 571 | 591 | GGTGCTCTGGCCGAGCATGGC | 156 |
| gctcggccagagcaccgagga | 73 | 576 | 596 | TCCTCGGTGCTCTGGCCGAGC | 157 |
| cctggcagtgtaccaggccgg | 74 | 675 | 695 | CCGGCCTGGTACACTGCCAGG | 158 |
| cctggggcccctggtggaaca | 75 | 741 | 761 | TGTTCCACCAGGGGCCCCAGG | 159 |
| ggcccctggtggaacagggcc | 76 | 746 | 766 | GGCCCTGTTCCACCAGGGGCC | 160 |
| ggatggaggagatgggcagcc | 77 | 851 | 871 | GGCTGCCCATCTCCTCCATCC | 161 |
| ccaagctggaggagcaggccc | 78 | 923 | 943 | GGGCCTGCTCCTCCAGCTTGG | 162 |
| gaggagcaggcccagcagata | 79 | 931 | 951 | TATCTGCTGGGCCTGCTCCTC | 163 |
| gcaggcccagcagatacgcct | 80 | 936 | 956 | AGGCGTATCTGCTGGGCCTGC | 164 |
| cccagcagatacgcctgcagg | 81 | 941 | 961 | CCTGCAGGCGTATCTGCTGGG | 165 |
| ccctggtggaagacatgcagc | 82 | 1001 | 1021 | GCTGCATGTCTTCCACCAGGG | 166 |
| gtggaagacatgcagcgccag | 83 | 1006 | 1026 | CTGGCGCTGCATGTCTTCCAC | 167 |
| agacatgcagcgccagtgggc | 84 | 1011 | 1031 | GCCCACTGGCGCTGCATGTCT | 168 |
| ggctggtggagaaggtgcag | 85 | 1034 | 1054 | CCTGCACCTTCTCCACCAGCC | 169 |
| gcctgcagccatgcgacccca | 86 | 1110 | 1130 | TGGGGTCGCATGGCTGCAGGC | 170 |

### Example 2

All oligonucleotides are synthesized on a 10-μmole scale using β-cyanoethylphosphoramidite chemistry on a solid support using automated DNA/RNA synthesizers (Mermade 6, BioAutomation, TX). The phosphoramidites of dA, dC, dG and dT and/or 2′-MOE modified A, C, G and T are sequentially coupled on desired sequences on an automated DNA/RNA synthesizer. The crude oligonucleotides are deprotected and cleaved from the solid support by treating concentrate ammonium hydroxide at 55° C. for overnight. The crude oligonucleotides are purified by a preparative anion exchange HPLC. The purified oligonucleotides are desalted from C₁₈ column and dialyzed against large volume of sterile water for overnight. Oligonucleotide solution is filtrated with a sterilized filter (0.2 μm or 0.45 μm HT Tuffryn Membrane, Pall Corporation) and then lyophilized for final product. All oligonucleotides are characterized by IE-HPLC (Waters 600, Waters 486 Tunable Absorbance Detector at 260 nm, Empower software) and MALDI-TOF mass spectrometry (Waters MALDI-ToF mass spectrometer with 337 nm N₂ laser) for purity and molecular mass, respectively. The purity of full-length oligonucleotides ranged from 95-98%, with the remainder lacking one or two nucleotides, as determined by ion-exchange HPLC.

To identify potent human ApoE antisense, a number of antisense oligonucleotides (ASOs) targeting human ApoE mRNA were screened in human Hep3B cells (ATCC, Manassas, Va.). 1×10⁵ cells were seeded in 24 well tissue culture plate and incubated overnight at 37° C., 5% CO₂. On the day of transfection, fresh medium was added to each well. Antisense oligonucleotides were prepared at 50 nM concentration in 50 μl serum free medium and mixed with 50 μl serum free medium containing 3 μl of lipofectamine 2000® (Thermo Fisher Scientific, Waltham, Mass.). The mixture was incubated at room temperature for 10 minutes and then applied to culture plates. Plates were then incubated for 48 hours at 37° C., 5% CO₂. Total RNA was isolated using RNAeasy Mini (Qiagen, Germantown, Md.) according to manufacturer's suggestion. RNA concentration was determined by UV spectrophotometer at 260/280 nm wavelength. For cDNA synthesis, 1 μg of total RNA was transcribed using High-Capacity cDNA Reverse Transcription kit (Thermo Fisher Scientific) according to manufacturer's suggestion. Human APOE mRNA expression level was determined by real-time quantitative PCR. Briefly, about 75 μg cDNA was mixed with 10 μl of TaqMan™ Fast Advanced Master Mix (Thermo Fisher Scientific) and 1 μl human APOE gene expression probe (Hs00171168_m1, Thermo Fisher Scientific) or 1 μl human HPRT1 gene expression probe (Hs02800695_m1, Thermo Fisher Scientific). Real-time quantitative PCR was performed using a StepOnePlus™ Real-Time PCR system (Thermo Fisher Scientific) and relative APOE gene expression was calculated using StepOne software version 2 (Thermo Fisher Scientific). Results are shown in Table 2.

TABLE 2
Human ApoE expression levels in
Hep3B cells treated with ASOs

| Human | % Human ApoE expression knock |
|---|---|
| ApoE ASO | down at 50 nM ASO concentration |
| SEQ ID NO: 87 | 31.5 |
| SEQ ID NO: 88 | 56.3 |
| SEQ ID NO: 89 | 39.1 |
| SEQ ID NO: 90 | 26.2 |
| SEQ ID NO: 91 | 41.4 |
| SEQ ID NO: 92 | 40.3 |
| SEQ ID NO: 93 | 26.7 |
| SEQ ID NO: 94 | 35.7 |
| SEQ ID NO: 95 | 36.3 |
| SEQ ID NO: 96 | 38.7 |
| SEQ ID NO: 97 | 37.9 |
| SEQ ID NO: 98 | 40.0 |
| SEQ ID NO: 99 | 22.4 |
| SEQ ID NO: 100 | 20.0 |
| SEQ ID NO: 101 | 18.5 |
| SEQ ID NO: 102 | 21.4 |
| SEQ ID NO: 103 | 34.3 |
| SEQ ID NO: 104 | 35.8 |
| SEQ ID NO: 105 | 45.7 |
| SEQ ID NO: 106 | 55.6 |
| SEQ ID NO: 107 | 60.2 |
| SEQ ID NO: 108 | 45.7 |
| SEQ ID NO: 109 | 44.3 |
| SEQ ID NO: 110 | 20.4 |
| SEQ ID NO: 111 | 55.1 |
| SEQ ID NO: 112 | 56.7 |
| SEQ ID NO: 113 | 60.5 |
| SEQ ID NO: 114 | 46.7 |
| SEQ ID NO: 115 | 54.4 |
| SEQ ID NO: 116 | 53.3 |
| SEQ ID NO: 117 | 58.7 |
| SEQ ID NO: 118 | 57.3 |
| SEQ ID NO: 119 | 20.2 |
| SEQ ID NO: 120 | 25.6 |
| SEQ ID NO: 121 | 67.9 |
| SEQ ID NO: 122 | 66.4 |
| SEQ ID NO: 123 | 54.1 |
| SEQ ID NO: 124 | 39.8 |
| SEQ ID NO: 125 | 38.2 |
| SEQ ID NO: 126 | 10.6 |
| SEQ ID NO: 127 | 5.7 |
| SEQ ID NO: 128 | 47.8 |
| SEQ ID NO: 129 | 76.0 |
| SEQ ID NO: 130 | 71.5 |
| SEQ ID NO: 131 | 67.0 |
| SEQ ID NO: 132 | 63.5 |
| SEQ ID NO: 133 | 71.0 |
| SEQ ID NO: 134 | 85.4 |
| SEQ ID NO: 135 | 84.5 |
| SEQ ID NO: 136 | 70.2 |
| SEQ ID NO: 137 | 76.6 |
| SEQ ID NO: 138 | 73.0 |
| SEQ ID NO: 139 | 70.7 |
| SEQ ID NO: 140 | 77.4 |
| SEQ ID NO: 141 | 65.4 |
| SEQ ID NO: 142 | 72.3 |
| SEQ ID NO: 143 | 70.5 |
| SEQ ID NO: 144 | 63.7 |
| SEQ ID NO: 145 | 67.8 |
| SEQ ID NO: 146 | 55.8 |
| SEQ ID NO: 147 | 59.9 |
| SEQ ID NO: 148 | 73.8 |
| SEQ ID NO: 149 | 52.3 |
| SEQ ID NO: 150 | 33.4 |
| SEQ ID NO: 151 | 27.6 |
| SEQ ID NO: 152 | 37.3 |
| SEQ ID NO: 153 | 38.8 |
| SEQ ID NO: 154 | 46.2 |
| SEQ ID NO: 155 | 63.2 |
| SEQ ID NO: 156 | 61.0 |
| SEQ ID NO: 157 | 66.4 |
| SEQ ID NO: 158 | 60.5 |
| SEQ ID NO: 159 | 69.5 |
| SEQ ID NO: 160 | 63.0 |
| SEQ ID NO: 161 | 61.3 |
| SEQ ID NO: 162 | 60.4 |
| SEQ ID NO: 163 | 57.7 |
| SEQ ID NO: 164 | 62.7 |
| SEQ ID NO: 165 | 48.6 |
| SEQ ID NO: 166 | 33.7 |
| SEQ ID NO: 167 | 23.0 |

To further characterize the impact of chemical modification of antisense oligos, 2′-MOE gapmers were synthesized as shown in Table 3.

TABLE 3
2′-MOE modified antisense oligonucleotides

| | Seq ID | Start | Stop | | Seq ID |
|---|---|---|---|---|---|
| Target sequence | No: | site | site | Antisense (5′-3′) | No: |
| ccagcagaccgagtggcagag | 45 | 246 | 266 | CTCTGCCACTCGGTCTGCTGG | 171 |
| tgggattacctgcgctgggtg | 49 | 301 | 321 | CACCCAGCGCAGGTAATCCCA | 172 |
| tgggtgcagacactgtctgag | 50 | 315 | 335 | TCAGACAGTGTCTGCACCCAG | 173 |
| tctgagcaggtgcaggaggag | 54 | 331 | 351 | CTCCTCCTGCACCTGCTCAGA | 174 |
| gaaggagttgaaggcctacaa | 64 | 405 | 425 | TTGTAGGCCTTCAACTCCTTC | 175 |
| gctcggccagagcaccgagga | 73 | 576 | 596 | TCCTCGGTGCTCTGGCCGAGC | 176 |
| gcaggcccagcagatacgcct | 80 | 936 | 956 | AGGCGTATCTGCTGGGCCTGC | 177 |

Underlined are 2′-MOE modified oligonucleotides

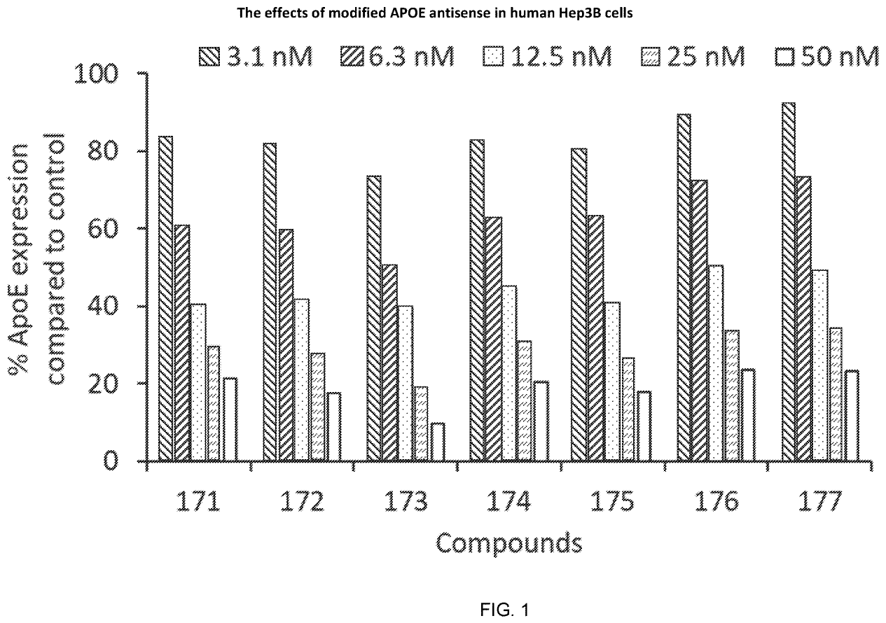

Human Hep3B cell lines (ATCC, Manassas, Va.) were used to assess human APOE mRNA expression. 1×10⁵ cells were seeded in 24 well tissue culture plate and incubated overnight at 37° C., 5% CO₂. On the day of transfection, fresh medium was added to each well. 2′-MOE modified antisense oligonucleotides were prepared at 3.2, 6.3, 12.5, 25 or 50 nM concentration in 50 μl serum free medium and mixed with 50 μl serum free medium containing 3 of lipofectamine 2000® (Thermo Fisher Scientific, Waltham, Mass.). The mixture was incubated at room temperature for 10 minutes and then applied to culture plates. Plates were then incubated for 48 hours at 37° C., 5% CO₂. Total RNA was isolated using RNAeasy Mini (Qiagen, Germantown, Md.) according to manufacturer's suggestion. RNA concentration was determined by UV spectrophotometer at 260/280 nm wavelength. For cDNA synthesis, 1 μg of total RNA was transcribed using High-Capacity cDNA Reverse Transcription kit (Thermo Fisher Scientific) according to manufacturer's suggestion. Human APOE mRNA expression level was determined by real-time quantitative PCR. Briefly, about 75 μg cDNA was mixed with 10 μl of TaqMan™ Fast Advanced Master Mix (Thermo Fisher Scientific) and 1 μl human APOE gene expression probe (Hs00171168_m1, Thermo Fisher Scientific) or 1 μl human HPRT1 gene expression probe (Hs02800695_m1, Thermo Fisher Scientific). Real-time quantitative PCR was performed using a StepOnePlus™ Real-Time PCR system (Thermo Fisher Scientific) and relative APOE gene expression was calculated using StepOne software version 2 (Thermo Fisher Scientific). Results shown in FIG. 1.

### REFERENCES

1. Hauser P S, Narayanaswami V, Ryan R O. Apolipoprotein E: from lipid transport to neurobiology. Prog Lipid Res. 2011; 50(1): 62-74.
2. Puglielli L, Tanzi R E, Kovacs D M. Alzheimer's disease: the cholesterol connection. Nat Neurosci. 2003; 6 (4): 345-51.
3. Mahley R W. Apolipoprotein E: from cardiovascular disease to neurodegenerative disorders. J Mol Med (Berl). 2016; 94(7): 739-46.
4. Liu C, Kanekiyo T, Xu H and Bu G. Apolipoprotein E and Alzheimer disease: risk, mechanisms, and therapy. Nat Rev Neurol. 2013; 9(2): 106-18.
5. Tenger C and Zhou X. Apolipoprotein E modulates immune activation by acting on the antigen-presenting cell. Immunology 2003; 109(3): 392-97.
6. Burgos J S, Ramirez C, Sastre I, Bullido M J, Valdivieso F. Involvement of apolipoprotein E in the hematogenous route of herpes simplex virus type 1 to the central nervous system. J Virol. 2002; 76(23): 12394-8.
7. Siddiqui R, Suzu S, Ueno M, Nasser H, Koba R, Bhuyan F, et al. Apolipoprotein E is an HIV-1-inducible inhibitor of viral production and infectivity in macrophages. PLoS Pathog. 2018; 14(11): e1007372.
8. Chang K S, Jiang J, Cai Z, Luo G. Human apolipoprotein e is required for infectivity and production of hepatitis C virus in cell culture. J Virol. 2007; 81(24): 13783-93.
9. Benga W J, Krieger S E, Dimitrova M, Zeisel M B, Parnot M, Lupberger J, et al. Apolipoprotein E interacts with hepatitis C virus nonstructural protein 5A and determines assembly of infectious particles. Hepatology. 2010; 51(1): 43-53.
10. Qiao L and Luo G. Human apolipoprotein E promotes hepatitis B virus infection and production. PLoS Pathog. 2019; 15(8): e1007874.
11. Shen Y, Li M, Ye X, Bi Q. Association of apolipoprotein E with the progression of hepatitis B virus-related liver disease. Int J Clin Exp Pathol. 2015 8(11): 14749-56.
12. Liang T J, Block T M, McMahon B J, Ghany M G, Urban S, Guo J T, et al. Present and future therapies of hepatitis B: From discovery to cure. Hepatology. 2015; 62(6): 1893-908.

While this invention has been particularly shown and described with references to preferred embodiments thereof, it will be understood by those skilled in the art that various changes in form and details may be made therein without departing from the scope of the invention encompassed by the appended claims.

1. A synthetic oligonucleotide compound comprising 12 to 30 phosphorothioate linked nucleotides having at least 12 contiguous nucleobases complementary to an equal length portion of SEQ ID NO: 1.

2. (canceled)

3. (canceled)

4. The compound according to claim 3, wherein the compound is at least 90% complementary over its entire length to the portion of SEQ ID NO: 1.

5. (canceled)

6. The compound according to claim 1, wherein the compound comprises at least 12 contiguous nucleobases of SEQ ID NO: 87-170, and is at least 90% complementary to its target sequence within SEQ ID NO: 1.

7-19. (canceled)

20. The compound according to claim 6, wherein the compound comprises SEQ ID NO: 87-170.

21. (canceled)

22. (canceled)

23. The compound according to claim 1, wherein the compound is at least 90% complementary to an equal length portion of a hotspot of SEQ ID NO: 1.

24. The compound according to claim 23, wherein the hotspot comprises nucleobases 200-600 or 900-1000 of SEQ ID NO: 1.

25. The compound according to claim 1, wherein the oligonucleotide compound has a gap segment; a 5′ wing segment; and a 3′ wing segment; wherein the gap segment is positioned between the 5′ wing segment and the 3′ wing segment, wherein each nucleoside of each wing segment comprises a comprises a modified nucleoside.

26. The compound according to claim 25, wherein the gap segment consisting of eleven linked deoxynucleosides; a 5′ wing segment consisting of five linked nucleosides; and a 3′ wing segment consisting of five linked nucleosides.

27. The compound according to claim 25, wherein the gap segment consisting of thirteen linked deoxynucleosides; a 5′ wing segment consisting of four linked nucleosides; and a 3′ wing segment consisting of four linked nucleosides.

28. The compound according to claim 1, wherein all of the nucleosides of the oligonucleotide compound comprise a modified nucleoside.

29. The compound according to claim 1, wherein the internucleoside linkages of the oligonucleotide compound is a phosphorothioate linkage, a phosphodiester linkage, or a combination thereof.

30. (canceled)

31. The compound according to claim 25, wherein the compound comprises at least 12 contiguous nucleobases of SEQ ID NO: 171-177, and is at least 90% complementary to its target sequence within SEQ ID NO: 1.

32. (canceled)

33. A composition comprising a compound according to claim 1 and a pharmaceutically acceptable carrier.

34. The composition according to claim 33, further comprising one or more vaccines, antigens, antibodies, cytotoxic agents, chemotherapeutic agents (both traditional chemotherapy and modern targeted therapies), radiation, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

35. A method for inhibiting Apolipoprotein E (ApoE) mRNA or protein expression, the method comprising contacting a cell with at least one compound according to claim 1 or a composition according to claim 33.

36. A method for treating a disease, disorder, or condition associated with ApoE expression and/or activity in an individual in need thereof, the method comprising administering at least one compound according to claim 1 or a composition according to claim 33.

37. The method according to claim 36, wherein the disease, disorder, or condition is selected from a liver disease or a neurological disease.

38. The method according to claim 37, wherein liver disease is hepatitis B.

39. The method according to claim 37, wherein the neurological disease is Alzheimer's.

40. The method according to claim 36, wherein the administering is by parenteral administration or local administration.

41. (canceled)

42. (canceled)

43. The method according to claim 36, wherein the method further comprises administering one or more vaccines, antigens, antibodies, cytotoxic agents, chemotherapeutic agents (both traditional chemotherapy and modern targeted therapies), radiation, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.


SEQUENCE LISTING (177 sequences)

SEQ ID NO: 1 - DNA, 1265 bp, Homo sapiens
  source 1..1265: mol_type = other DNA
ggatggggagataagagaagaccaggagggagttaaatagggaatgggttgggggcggcttggtaaatgtgctgggattaggctgttgcagataatgcaacaaggcttggaaggctaacctgggactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatcaaaaaaaaaaaaaaaaaaa

SEQ ID NO: 2 - DNA, 1358 bp, Mus musculus
  source 1..1358: mol_type = other DNA
tttcctctgccctgctgtgaagggggagagaacaacccgcctcgtgacagggggctggcacagcccgccctagccctgaggagggggcgggacagggggagtcctataattggaccggtctgggatccgatcccctgctcagaccctggaggctaaggacttgtttcggaaggagctggagagggagctggaatttttggcagcggatccaccccggggtgccgagatagcgaactcggcaaggggagactggccaatcacaattgcgaagatgaaggctctgtgggccgtgctgttggtcacattgctgacaggatgcctagccgagggagagccggaggtgacagatcagctcgagtggcaaagcaaccaaccctgggagcaggccctgaaccgcttctgggattacctgcgctgggtgcagacgctgtctgaccaggtccaggaagagctgcagagctcccaagtcacacaagaactgacggcactgatggaggacactatgacggaagtaaaggcttacaaaaaggagctggaggaacagctgggtccagtggcggaggagacacgggccaggctgggcaaagaggtgcaggcggcacaggcccgactcggagccgacatggaggatctacgcaaccgactcgggcagtaccgcaacgaggtgcacaccatgctgggccagagcacagaggagatacgggcgcggctctccacacacctgcgcaagatgcgcaagcgcttgatgcgggatgccgaggatctgcagaagcgcctagctgtgtacaaggcaggggcacgcgagggcgccgagcgcggtgtgagtgccatccgtgagcgcctggggcctctggtggagcaaggtcgccagcgcactgccaacctaggcgctggggccgcccagcctctgcgcgatcgcgcccaggcttttggtgaccgcatccgagggcggctggaggaagtgggcaaccaggcccgtgaccgcctagaggaggtgcgtgagcacatggaggaggtgcgctccaagatggaggaacagacccagcaaatacgcctgcaggcggagatcttccaggcccgcctcaagggctggttcgagccaatagtggaagacatgcatcgccagtgggcaaacctgatggagaagatacaggcctctgtggctaccaaccccatcatcaccccagtggcccaggagaatcaatgagtatccttctcctgtcctgcaacaacatccatatccagccaggtggccctgtctcaagcacctctctggccctctggtggcccttgcttaataaagattctccgagcacattctgagtctctgtgagtgattccaaaaaaaaaaaaaaaaa

SEQ ID NO: 3 - DNA, 21 bp, synthetic construct
  source 1..21: mol_type = other DNA
ggatggggagataagagaaga

SEQ ID NO: 4 - DNA, 21 bp, synthetic construct
  source 1..21: mol_type = other DNA
gggagataagagaagaccagg

SEQ ID NO: 5 - DNA, 21 bp, synthetic construct
  source 1..21: mol_type = other DNA
ataagagaagaccaggaggga

SEQ ID NO: 6 - DNA, 21 bp, synthetic construct
  source 1..21: mol_type = other DNA
agaagaccaggagggagttaa

SEQ ID NO: 7 - DNA, 21 bp, synthetic construct
  source 1..21: mol_type = other DNA
gaagaccaggagggagttaaa

SEQ ID NO: 8 - DNA, 21 bp, synthetic construct
  source 1..21: mol_type = other DNA
accaggagggagttaaatagg

SEQ ID NO: 9 - DNA, 21 bp, synthetic construct
  source 1..21: mol_type = other DNA
gagggagttaaatagggaatg

SEQ ID NO: 10 - DNA, 21 bp, synthetic construct
  source 1..21: mol_type = other DNA
agttaaatagggaatgggttg

SEQ ID NO: 11 - DNA, 21 bp, synthetic construct
  source 1..21: mol_type = other DNA
aatagggaatgggttgggggc

SEQ ID NO: 12 - DNA, 21 bp, synthetic construct
  source 1..21: mol_type = other DNA
ggaatgggttgggggcggctt

SEQ ID NO: 13 - DNA, 21 bp, synthetic construct
  source 1..21: mol_type = other DNA
cggcttggtaaatgtgctggg

SEQ ID NO: 14 - DNA, 21 bp, synthetic construct
  source 1..21: mol_type = other DNA
tggtaaatgtgctgggattag

SEQ ID NO: 15 - DNA, 21 bp, synthetic construct
  source 1..21: mol_type = other DNA
aatgtgctgggattaggctgt

SEQ ID NO: 16 - DNA, 21 bp, synthetic construct
  source 1..21: mol_type = other DNA
gctgggattaggctgttgcag

SEQ ID NO: 17 - DNA, 21 bp, synthetic construct
  source 1..21: mol_type = other DNA
ggattaggctgttgcagataa

SEQ ID NO: 18 - DNA, 21 bp, synthetic construct
  source 1..21: mol_type = other DNA
gattaggctgttgcagataat

SEQ ID NO: 19 - DNA, 21 bp, synthetic construct
  source 1..21: mol_type = other DNA
ggctgttgcagataatgcaac

SEQ ID NO: 20 - DNA, 21 bp, synthetic construct
  source 1..21: mol_type = other DNA
ttgcagataatgcaacaaggc

SEQ ID NO: 21 - DNA, 21 bp, synthetic construct
  source 1..21: mol_type = other DNA
gataatgcaacaaggcttgga

SEQ ID NO: 22 - DNA, 21 bp, synthetic construct
  source 1..21: mol_type = other DNA
tgcaacaaggcttggaaggct

SEQ ID NO: 23 - DNA, 21 bp, synthetic construct
  source 1..21: mol_type = other DNA
caaggcttggaaggctaacct

SEQ ID NO: 24 - DNA, 21 bp, synthetic construct
  source 1..21: mol_type = other DNA
cttggaaggctaacctgggac

SEQ ID NO: 25 - DNA, 21 bp, synthetic construct
  source 1..21: mol_type = other DNA
aaggctaacctgggactggcc

SEQ ID NO: 26 - DNA, 21 bp, synthetic construct
  source 1..21: mol_type = other DNA
taacctgggactggccaatca

SEQ ID NO: 27 - DNA, 21 bp, synthetic construct
  source 1..21: mol_type = other DNA
tgggactggccaatcacaggc

SEQ ID NO: 28 - DNA, 21 bp, synthetic construct
  source 1..21: mol_type = other DNA
ctggccaatcacaggcaggaa

SEQ ID NO: 29 - DNA, 21 bp, synthetic construct
  source 1..21: mol_type = other DNA
caatcacaggcaggaagatga

SEQ ID NO: 30 - DNA, 21 bp, synthetic construct
  source 1..21: mol_type = other DNA
acaggcaggaagatgaaggtt

SEQ ID NO: 31 - DNA, 21 bp, synthetic construct
  source 1..21: mol_type = other DNA
gcaggaagatgaaggttctgt

SEQ ID NO: 32 - DNA, 21 bp, synthetic construct
  source 1..21: mol_type = other DNA
caggaagatgaaggttctgtg

SEQ ID NO: 33 - DNA, 21 bp, synthetic construct
  source 1..21: mol_type = other DNA
gaagatgaaggttctgtgggc

SEQ ID NO: 34 - DNA, 21 bp, synthetic construct
  source 1..21: mol_type = other DNA
agatgaaggttctgtgggctg

SEQ ID NO: 35 - DNA, 21 bp, synthetic construct
  source 1..21: mol_type = other DNA
gaaggttctgtgggctgcgtt

SEQ ID NO: 36 - DNA, 21 bp, synthetic construct
  source 1..21: mol_type = other DNA
gcgttgctggtcacattcctg

SEQ ID NO: 37 - DNA, 21 bp, synthetic construct
  source 1..21: mol_type = other DNA
gctggtcacattcctggcagg

SEQ ID NO: 38 - DNA, 21 bp, synthetic construct
  source 1..21: mol_type = other DNA
tcacattcctggcaggatgcc

SEQ ID NO: 39 - DNA, 21 bp, synthetic construct
  source 1..21: mol_type = other DNA
ttcctggcaggatgccaggcc

SEQ ID NO: 40 - DNA, 21 bp, synthetic construct
  source 1..21: mol_type = other DNA
ggcaggatgccaggccaaggt

SEQ ID NO: 41 - DNA, 21 bp, synthetic construct
  source 1..21: mol_type = other DNA
gatgccaggccaaggtggagc

SEQ ID NO: 42 - DNA, 21 bp, synthetic construct
  source 1..21: mol_type = other DNA
caggccaaggtggagcaagcg

SEQ ID NO: 43 - DNA, 21 bp, synthetic construct
  source 1..21: mol_type = other DNA
ggtggagacagagccggagcc

SEQ ID NO: 44 - DNA, 21 bp, synthetic construct
  source 1..21: mol_type = other DNA
ctgcgccagcagaccgagtgg

SEQ ID NO: 45 - DNA, 21 bp, synthetic construct
  source 1..21: mol_type = other DNA
ccagcagaccgagtggcagag

SEQ ID NO: 46 - DNA, 21 bp, synthetic construct
  source 1..21: mol_type = other DNA
gccagcgctgggaactggcac

SEQ ID NO: 47 - DNA, 21 bp, synthetic construct
  source 1..21: mol_type = other DNA
ctgggaactggcactgggtcg

SEQ ID NO: 48 - DNA, 21 bp, synthetic construct
  source 1..21: mol_type = other DNA
gcttttgggattacctgcgct

SEQ ID NO: 49 - DNA, 21 bp, synthetic construct
  source 1..21: mol_type = other DNA
tgggattacctgcgctgggtg

SEQ ID NO: 50 - DNA, 21 bp, synthetic construct
  source 1..21: mol_type = other DNA
ctgggtgcagacactgtctga

SEQ ID NO: 51 - DNA, 21 bp, synthetic construct
  source 1..21: mol_type = other DNA
tgggtgcagacactgtctgag

SEQ ID NO: 52 - DNA, 21 bp, synthetic construct
  source 1..21: mol_type = other DNA
gcagacactgtctgagcaggt

SEQ ID NO: 53 - DNA, 21 bp, synthetic construct
  source 1..21: mol_type = other DNA
cactgtctgagcaggtgcagg

SEQ ID NO: 54 - DNA, 21 bp, synthetic construct
  source 1..21: mol_type = other DNA
tctgagcaggtgcaggaggag

SEQ ID NO: 55 - DNA, 21 bp, synthetic construct
  source 1..21: mol_type = other DNA
gcaggtgcaggaggagctgct

SEQ ID NO: 56 - DNA, 21 bp, synthetic construct
  source 1..21: mol_type = other DNA
tgcaggaggagctgctcagct

SEQ ID NO: 57 - DNA, 21 bp, synthetic construct
  source 1..21: mol_type = other DNA
gaggagctgctcagctcccag

SEQ ID NO: 58 - DNA, 21 bp, synthetic construct
  source 1..21: mol_type = other DNA
gctgctcagctcccaggtcac

SEQ ID NO: 59 - DNA, 21 bp, synthetic construct
  source 1..21: mol_type = other DNA
tcagctcccaggtcacccagg

SEQ ID NO: 60 - DNA, 21 bp, synthetic construct
  source 1..21: mol_type = other DNA
tcccaggtcacccaggaactg

SEQ ID NO: 61 - DNA, 21 bp, synthetic construct
  source 1..21: mol_type = other DNA
aggtcacccaggaactgaggg

SEQ ID NO: 62 - DNA, 21 bp, synthetic construct
  source 1..21: mol_type = other DNA
ggacgagaccatgaaggagtt

SEQ ID NO: 63 - DNA, 21 bp, synthetic construct
  source 1..21: mol_type = other DNA
ccatgaaggagttgaaggcct

SEQ ID NO: 64 - DNA, 21 bp, synthetic construct
  source 1..21: mol_type = other DNA
gaaggagttgaaggcctacaa

SEQ ID NO: 65 - DNA, 21 bp, synthetic construct
  source 1..21: mol_type = other DNA
aaggagttgaaggcctacaaa

SEQ ID NO: 66 - DNA, 21 bp, synthetic construct
  source 1..21: mol_type = other DNA
gttgaaggcctacaaatcgga

SEQ ID NO: 67 - DNA, 21 bp, synthetic construct
  source 1..21: mol_type = other DNA
aactggaggaacaactgaccc

SEQ ID NO: 68 - DNA, 21 bp, synthetic construct
  source 1..21: mol_type = other DNA
ggaggaacaactgaccccggt

SEQ ID NO: 69 - DNA, 21 bp, synthetic construct
  source 1..21: mol_type = other DNA
ctgaccccggtggcggaggac

SEQ ID NO: 70 - DNA, 21 bp, synthetic construct
  source 1..21: mol_type = other DNA
ggctgtccaaggagctgcagg

SEQ ID NO: 71 - DNA, 21 bp, synthetic construct
  source 1..21: mol_type = other DNA
aggtgcaggccatgctcggcc

SEQ ID NO: 72 - DNA, 21 bp, synthetic construct
  source 1..21: mol_type = other DNA
gccatgctcggccagagcacc

SEQ ID NO: 73 - DNA, 21 bp, synthetic construct
  source 1..21: mol_type = other DNA
gctcggccagagcaccgagga

SEQ ID NO: 74 - DNA, 21 bp, synthetic construct
  source 1..21: mol_type = other DNA
cctggcagtgtaccaggccgg

SEQ ID NO: 75 - DNA, 21 bp, synthetic construct
  source 1..21: mol_type = other DNA
cctggggcccctggtggaaca

SEQ ID NO: 76 - DNA, 21 bp, synthetic construct
  source 1..21: mol_type = other DNA
ggcccctggtggaacagggcc

SEQ ID NO: 77 - DNA, 21 bp, synthetic construct
  source 1..21: mol_type = other DNA
ggatggaggagatgggcagcc

SEQ ID NO: 78 - DNA, 21 bp, synthetic construct
  source 1..21: mol_type = other DNA
ccaagctggaggagcaggccc

SEQ ID NO: 79 - DNA, 21 bp, synthetic construct
  source 1..21: mol_type = other DNA
gaggagcaggcccagcagata

SEQ ID NO: 80 - DNA, 21 bp, synthetic construct
  source 1..21: mol_type = other DNA
gcaggcccagcagatacgcct

SEQ ID NO: 81 - DNA, 21 bp, synthetic construct
  source 1..21: mol_type = other DNA
cccagcagatacgcctgcagg

SEQ ID NO: 82 - DNA, 21 bp, synthetic construct
  source 1..21: mol_type = other DNA
ccctggtggaagacatgcagc

SEQ ID NO: 83 - DNA, 21 bp, synthetic construct
  source 1..21: mol_type = other DNA
gtggaagacatgcagcgccag

SEQ ID NO: 84 - DNA, 21 bp, synthetic construct
  source 1..21: mol_type = other DNA
agacatgcagcgccagtgggc

SEQ ID NO: 85 - DNA, 20 bp, synthetic construct
  source 1..20: mol_type = other DNA
ggctggtggagaaggtgcag

SEQ ID NO: 86 - DNA, 21 bp, synthetic construct
  source 1..21: mol_type = other DNA
gcctgcagccatgcgacccca

SEQ ID NO: 87 - DNA, 21 bp, synthetic construct
  source 1..21: mol_type = other DNA
tcttctcttatctccccatcc

SEQ ID NO: 88 - DNA, 21 bp, synthetic construct
  source 1..21: mol_type = other DNA
cctggtcttctcttatctccc

SEQ ID NO: 89 - DNA, 21 bp, synthetic construct
  source 1..21: mol_type = other DNA
tccctcctggtcttctcttat

SEQ ID NO: 90 - DNA, 21 bp, synthetic construct
  source 1..21: mol_type = other DNA
ttaactccctcctggtcttct

SEQ ID NO: 91 - DNA, 21 bp, synthetic construct
  source 1..21: mol_type = other DNA
tttaactccctcctggtcttc

SEQ ID NO: 92 - DNA, 21 bp, synthetic construct
  source 1..21: mol_type = other DNA
cctatttaactccctcctggt

SEQ ID NO: 93 - DNA, 21 bp, synthetic construct
  source 1..21: mol_type = other DNA
cattccctatttaactccctc

SEQ ID NO: 94 - DNA, 21 bp, synthetic construct
  source 1..21: mol_type = other DNA
caacccattccctatttaact

SEQ ID NO: 95 - DNA, 21 bp, synthetic construct
  source 1..21: mol_type = other DNA
gcccccaacccattccctatt

SEQ ID NO: 96 - DNA, 21 bp, synthetic construct
  source 1..21: mol_type = other DNA
aagccgcccccaacccattcc

SEQ ID NO: 97 - DNA, 21 bp, synthetic construct
  source 1..21: mol_type = other DNA
cccagcacatttaccaagccg

SEQ ID NO: 98 - DNA, 21 bp, synthetic construct
  source 1..21: mol_type = other DNA
ctaatcccagcacatttacca

SEQ ID NO: 99 - DNA, 21 bp, synthetic construct
  source 1..21: mol_type = other DNA
acagcctaatcccagcacatt

SEQ ID NO: 100 - DNA, 21 bp, synthetic construct
  source 1..21: mol_type = other DNA
ctgcaacagcctaatcccagc

SEQ ID NO: 101 - DNA, 21 bp, synthetic construct
  source 1..21: mol_type = other DNA
ttatctgcaacagcctaatcc

SEQ ID NO: 102 - DNA, 21 bp, synthetic construct
  source 1..21: mol_type = other DNA
attatctgcaacagcctaatc

SEQ ID NO: 103 - DNA, 21 bp, synthetic construct
  source 1..21: mol_type = other DNA
gttgcattatctgcaacagcc

SEQ ID NO: 104 - DNA, 21 bp, synthetic construct
  source 1..21: mol_type = other DNA
gccttgttgcattatctgcaa

SEQ ID NO: 105 - DNA, 21 bp, synthetic construct
  source 1..21: mol_type = other DNA
tccaagccttgttgcattatc

SEQ ID NO: 106 - DNA, 21 bp, synthetic construct
  source 1..21: mol_type = other DNA
agccttccaagccttgttgca

SEQ ID NO: 107 - DNA, 21 bp, synthetic construct
  source 1..21: mol_type = other DNA
aggttagccttccaagccttg

SEQ ID NO: 108 - DNA, 21 bp, synthetic construct
  source 1..21: mol_type = other DNA
gtcccaggttagccttccaag

SEQ ID NO: 109 - DNA, 21 bp, synthetic construct
  source 1..21: mol_type = other DNA
ggccagtcccaggttagcctt

SEQ ID NO: 110 - DNA, 21 bp, synthetic construct
  source 1..21: mol_type = other DNA
tgattggccagtcccaggtta

SEQ ID NO: 111 - DNA, 21 bp, synthetic construct
  source 1..21: mol_type = other DNA
gcctgtgattggccagtccca

SEQ ID NO: 112 - DNA, 21 bp, synthetic construct
  source 1..21: mol_type = other DNA
ttcctgcctgtgattggccag

SEQ ID NO: 113 - DNA, 21 bp, synthetic construct
  source 1..21: mol_type = other DNA
tcatcttcctgcctgtgattg

SEQ ID NO: 114 - DNA, 21 bp, synthetic construct
  source 1..21: mol_type = other DNA
aaccttcatcttcctgcctgt

SEQ ID NO: 115 - DNA, 21 bp, synthetic construct
  source 1..21: mol_type = other DNA
acagaaccttcatcttcctgc

SEQ ID NO: 116 - DNA, 21 bp, synthetic construct
  source 1..21: mol_type = other DNA
cacagaaccttcatcttcctg

SEQ ID NO: 117 - DNA, 21 bp, synthetic construct
  source 1..21: mol_type = other DNA
gcccacagaaccttcatcttc

SEQ ID NO: 118 - DNA, 21 bp, synthetic construct
  source 1..21: mol_type = other DNA
cagcccacagaaccttcatct

SEQ ID NO: 119 - DNA, 21 bp, synthetic construct
  source 1..21: mol_type = other DNA
aacgcagcccacagaaccttc

SEQ ID NO: 120 - DNA, 21 bp, synthetic construct
  source 1..21: mol_type = other DNA
caggaatgtgaccagcaacgc

SEQ ID NO: 121 - DNA, 21 bp, synthetic construct
  source 1..21: mol_type = other DNA
cctgccaggaatgtgaccagc

SEQ ID NO: 122 - DNA, 21 bp, synthetic construct
  source 1..21: mol_type = other DNA
ggcatcctgccaggaatgtga

SEQ ID NO: 123 - DNA, 21 bp, synthetic construct
  source 1..21: mol_type = other DNA
ggcctggcatcctgccaggaa

SEQ ID NO: 124 - DNA, 21 bp, synthetic construct
  source 1..21: mol_type = other DNA
accttggcctggcatcctgcc

SEQ ID NO: 125 - DNA, 21 bp, synthetic construct
  source 1..21: mol_type = other DNA
gctccaccttggcctggcatc

SEQ ID NO: 126 - DNA, 21 bp, synthetic construct
  source 1..21: mol_type = other DNA
cgcttgctccaccttggcctg

SEQ ID NO: 127 - DNA, 21 bp, synthetic construct
  source 1..21: mol_type = other DNA
ggctccggctctgtctccacc

SEQ ID NO: 128 - DNA, 21 bp, synthetic construct
  source 1..21: mol_type = other DNA
ccactcggtctgctggcgcag

SEQ ID NO: 129 - DNA, 21 bp, synthetic construct
  source 1..21: mol_type = other DNA
ctctgccactcggtctgctgg

SEQ ID NO: 130 - DNA, 21 bp, synthetic construct
  source 1..21: mol_type = other DNA
gtgccagttcccagcgctggc

SEQ ID NO: 131 - DNA, 21 bp, synthetic construct
  source 1..21: mol_type = other DNA
cgacccagtgccagttcccag

SEQ ID NO: 132 - DNA, 21 bp, synthetic construct
  source 1..21: mol_type = other DNA
agcgcaggtaatcccaaaagc

SEQ ID NO: 133 - DNA, 21 bp, synthetic construct
  source 1..21: mol_type = other DNA
cacccagcgcaggtaatccca

SEQ ID NO: 134 - DNA, 21 bp, synthetic construct
  source 1..21: mol_type = other DNA
tcagacagtgtctgcacccag

SEQ ID NO: 135 - DNA, 21 bp, synthetic construct
  source 1..21: mol_type = other DNA
ctcagacagtgtctgcaccca

SEQ ID NO: 136 - DNA, 21 bp, synthetic construct
  source 1..21: mol_type = other DNA
acctgctcagacagtgtctgc

SEQ ID NO: 137 - DNA, 21 bp, synthetic construct
  source 1..21: mol_type = other DNA
cctgcacctgctcagacagtg

SEQ ID NO: 138 - DNA, 21 bp, synthetic construct
  source 1..21: mol_type = other DNA
ctcctcctgcacctgctcaga

SEQ ID NO: 139 - DNA, 21 bp, synthetic construct
  source 1..21: mol_type = other DNA
agcagctcctcctgcacctgc

SEQ ID NO: 140 - DNA, 21 bp, synthetic construct
  source 1..21: mol_type = other DNA
agctgagcagctcctcctgca

SEQ ID NO: 141 - DNA, 21 bp, synthetic construct
  source 1..21: mol_type = other DNA
ctgggagctgagcagctcctc

SEQ ID NO: 142 - DNA, 21 bp, synthetic construct
  source 1..21: mol_type = other DNA
gtgacctgggagctgagcagc

SEQ ID NO: 143 - DNA, 21 bp, synthetic construct
  source 1..21: mol_type = other DNA
cctgggtgacctgggagctga

SEQ ID NO: 144 - DNA, 21 bp, synthetic construct
  source 1..21: mol_type = other DNA
cagttcctgggtgacctggga

SEQ ID NO: 145 - DNA, 21 bp, synthetic construct
  source 1..21: mol_type = other DNA
ccctcagttcctgggtgacct

SEQ ID NO: 146 - DNA, 21 bp, synthetic construct
  source 1..21: mol_type = other DNA
aactccttcatggtctcgtcc

SEQ ID NO: 147 - DNA, 21 bp, synthetic construct
  source 1..21: mol_type = other DNA
aggccttcaactccttcatgg

SEQ ID NO: 148 - DNA, 21 bp, synthetic construct
  source 1..21: mol_type = other DNA
ttgtaggccttcaactccttc

SEQ ID NO: 149 - DNA, 21 bp, synthetic construct
  source 1..21: mol_type = other DNA
tttgtaggccttcaactcctt

SEQ ID NO: 150 - DNA, 21 bp, synthetic construct
  source 1..21: mol_type = other DNA
tccgatttgtaggccttcaac

SEQ ID NO: 151 - DNA, 21 bp, synthetic construct
  source 1..21: mol_type = other DNA
gggtcagttgttcctccagtt

SEQ ID NO: 152 - DNA, 21 bp, synthetic construct
  source 1..21: mol_type = other DNA
accggggtcagttgttcctcc

SEQ ID NO: 153 - DNA, 21 bp, synthetic construct
  source 1..21: mol_type = other DNA
gtcctccgccaccggggtcag

SEQ ID NO: 154 - DNA, 21 bp, synthetic construct
  source 1..21: mol_type = other DNA
cctgcagctccttggacagcc

SEQ ID NO: 155 - DNA, 21 bp, synthetic construct
  source 1..21: mol_type = other DNA
ggccgagcatggcctgcacct

SEQ ID NO: 156 - DNA, 21 bp, synthetic construct
  source 1..21: mol_type = other DNA
ggtgctctggccgagcatggc

SEQ ID NO: 157 - DNA, 21 bp, synthetic construct
  source 1..21: mol_type = other DNA
tcctcggtgctctggccgagc

SEQ ID NO: 158 - DNA, 21 bp, synthetic construct
  source 1..21: mol_type = other DNA
ccggcctggtacactgccagg

SEQ ID NO: 159 - DNA, 21 bp, synthetic construct
  source 1..21: mol_type = other DNA
tgttccaccaggggccccagg

SEQ ID NO: 160 - DNA, 21 bp, synthetic construct
  source 1..21: mol_type = other DNA
ggccctgttccaccaggggcc

SEQ ID NO: 161 - DNA, 21 bp, synthetic construct
  source 1..21: mol_type = other DNA
ggctgcccatctcctccatcc

SEQ ID NO: 162 - DNA, 21 bp, synthetic construct
  source 1..21: mol_type = other DNA
gggcctgctcctccagcttgg

SEQ ID NO: 163 - DNA, 21 bp, synthetic construct
  source 1..21: mol_type = other DNA
tatctgctgggcctgctcctc

SEQ ID NO: 164 - DNA, 21 bp, synthetic construct
  source 1..21: mol_type = other DNA
aggcgtatctgctgggcctgc

SEQ ID NO: 165 - DNA, 21 bp, synthetic construct
  source 1..21: mol_type = other DNA
cctgcaggcgtatctgctggg

SEQ ID NO: 166 - DNA, 21 bp, synthetic construct
  source 1..21: mol_type = other DNA
gctgcatgtcttccaccaggg

SEQ ID NO: 167 - DNA, 21 bp, synthetic construct
  source 1..21: mol_type = other DNA
ctggcgctgcatgtcttccac

SEQ ID NO: 168 - DNA, 21 bp, synthetic construct
  source 1..21: mol_type = other DNA
gcccactggcgctgcatgtct

SEQ ID NO: 169 - DNA, 21 bp, synthetic construct
  source 1..21: mol_type = other DNA
cctgcaccttctccaccagcc

SEQ ID NO: 170 - DNA, 21 bp, synthetic construct
  source 1..21: mol_type = other DNA
tggggtcgcatggctgcaggc

SEQ ID NO: 171 - DNA, 21 bp, synthetic construct
  misc_feature 1..5: note = 2'-MOE
  misc_feature 17..21: note = 2'-MOE
  source 1..21: mol_type = other DNA
ctctgccactcggtctgctgg

SEQ ID NO: 172 - DNA, 21 bp, synthetic construct
  misc_feature 1..5: note = 2'-MOE
  misc_feature 17..21: note = 2'-MOE
  source 1..21: mol_type = other DNA
cacccagcgcaggtaatccca

SEQ ID NO: 173 - DNA, 21 bp, synthetic construct
  misc_feature 1..5: note = 2'-MOE
  misc_feature 17..21: note = 2'-MOE
  source 1..21: mol_type = other DNA
tcagacagtgtctgcacccag

SEQ ID NO: 174 - DNA, 21 bp, synthetic construct
  misc_feature 1..5: note = 2'-MOE
  misc_feature 17..21: note = 2'-MOE
  source 1..21: mol_type = other DNA
ctcctcctgcacctgctcaga

SEQ ID NO: 175 - DNA, 21 bp, synthetic construct
  misc_feature 1..5: note = 2'-MOE
  misc_feature 17..21: note = 2'-MOE
  source 1..21: mol_type = other DNA
ttgtaggccttcaactccttc

SEQ ID NO: 176 - DNA, 21 bp, synthetic construct
  misc_feature 1..5: note = 2'-MOE
  misc_feature 17..21: note = 2'-MOE
  source 1..21: mol_type = other DNA
tcctcggtgctctggccgagc

SEQ ID NO: 177 - DNA, 21 bp, synthetic construct
  misc_feature 1..5: note = 2'-MOE
  misc_feature 17..21: note = 2'-MOE
  source 1..21: mol_type = other DNA
aggcgtatctgctgggcctgc